(19) United States
(12) Patent Application Publication
(10) Pub. No.: US 2026/0157875 A1
(43) Pub. Date: Jun. 11, 2026

(54) MULTI-MODE PORTABLE EVAPORATIVE COOLER

(71) Applicant: Rex A. Eiserer, Austin, TX (US)

(72) Inventor: Rex A. Eiserer, Austin, TX (US)

(21) Appl. No.: 19/394,279

(22) Filed: Nov. 19, 2025

Related U.S. Application Data
(60) Provisional application No. 63/722,377, filed on Nov. 19, 2024.
(60) Provisional application No. 63/783,095, filed on Apr. 3, 2025.

(51) Int. Cl. A61F 7/00

(52) U.S. Cl. CPC A61F 7/0053; A61F 2007/006; A61F 2007/0068

(57) ABSTRACT

A multi-mode evaporative cooling system operates alternately in a first mode to provide cooled, moist air via internal evaporation, and a second mode for cooling an object (e.g., a body) via conduction. In the second mode, the cooler suspends internal evaporation to direct warm, dry air toward a wetted object, causing water on the object to evaporate, which provides more efficient cooling useful for the treatment of heat injuries. The system may be medialess to allow the suspension of internal evaporation to occur quickly. The system includes a fan and may utilize a control system coupled to sensors and components to regulate operation.

### CROSS-REFERENCE TO RELATED APPLICATION(S)

This application claims the benefit of U.S. Provisional Application No. 63/722,377, filed Nov. 19, 2024, entitled “MULTI-MODE PORTABLE EVAPORATIVE COOLER,” and U.S. Provisional Application No. 63/783,095, filed Apr. 3, 2025, entitled “MULTI-MODE PORTABLE EVAPORATIVE COOLER SYSTEM”. The contents of both U.S. Provisional Application No. 63/722,377 and U.S. Provisional Application No. 63/783,095 are incorporated herein by reference in their entireties.

### TECHNICAL FIELD

The invention relates generally to evaporative cooling systems and more particularly to multi-mode evaporative cooling systems. These systems may include those that are medialess, those having housings transformable from a compact storage configuration to a larger operating configuration, and those utilizing control systems for controlling operation.

### BACKGROUND

Conventional evaporative cooling systems have been available for many years. These systems operate primarily in one mode (purpose), which is providing cooled air for comfort or relief from heat through convection cooling. This involves evaporating water inside the cooler to cool the air, and then directing the cooled air to an area to cool objects or people. Typically, these systems operate by directing water onto an evaporative cooling medium (media) and directing air over the medium, where evaporation cools the air.

A significant challenge with most conventional evaporative coolers is that while they may have the ability to stop the flow or circulation of water across evaporative media, this does not necessarily stop the evaporation process immediately. Some water remains on the evaporative media, and evaporation continues until the water is completely evaporated. As long as this evaporation continues, the air flowing through the cooler will continue to be cooled and moisture will be added to the air. Both of these factors—the continued cooling and moisture addition—reduce the ability of the air exiting the cooler to evaporate water from a body located outside the cooler. Furthermore, conventional portable evaporative cooling systems often have large, rigid shrouds. This results in a relatively large amount of space required for storage and transport, which is a significant disadvantage particularly for systems intended to be transportable. The effectiveness of these conventional systems is also dependent upon the humidity of the air in the area in which they are used, with higher humidity reducing cooling effectiveness.

Therefore, there is a need for a portable evaporative cooler that provides multiple operating modes, including one that enables the rapid suspension of internal evaporation to facilitate the efficient treatment of heat injuries via external conductive cooling.

### SUMMARY

Embodiments of a portable evaporative cooler that can be used to both prevent and treat heat injuries with unique modes of operation are disclosed. Embodiments of disclosed coolers have two modes of operation. A first mode relates to convective cooling. In this mode, water is evaporated within the cooler enclosure to cool incoming dry, warm air. The air exiting the cooler in this mode is cool and moist. A second mode relates to conductive cooling/artificial sweating. Embodiments of this mode are adapted to cool an object, such as a body, by conduction. In some embodiments, this mode involves two parts: first, the evaporation process inside the cooler enclosure is suspended or avoided, allowing warm, unsaturated air to enter and exit the cooler; second, water is applied to the object (body), and the exiting warm, dry air is directed over the wetted object to evaporate the water off the object.

The second mode is particularly useful for the treatment of heat injuries because conductive cooling (evaporating water off the body) is a more efficient and effective means to cool the object than convective cooling (directing already-cooled air over the body). This process can be loosely described as artificial sweating.

To facilitate the quick transition necessary for emergency treatment, some embodiments employ a medialess evaporative cooler. Since there is no evaporative medium, the internal evaporation process can be stopped quickly, providing an advantage over traditional media-based coolers. The system may include a control system that receives inputs selecting the mode of operation and directs water flow accordingly. Furthermore, the system enclosure may have a portion that is alternately expandable and contractible, enabling the system to occupy a reduced volume for easier storage and transportation.

Embodiments of the present invention also include computer-readable storage media containing sets of instructions to cause one or more processors to perform the methods, variations of the methods, and other operations described herein.

These, and other, aspects of the disclosure will be better appreciated and understood when considered in conjunction with the following description and the accompanying drawings. It should be understood, however, that the following description, while indicating various embodiments of the disclosure and numerous specific details thereof, is given by way of illustration and not of limitation. Many substitutions, modifications, additions and/or rearrangements may be made within the scope of the disclosure without departing from the spirit thereof, and the disclosure includes all such substitutions, modifications, additions and/or rearrangements.

### BRIEF DESCRIPTION OF THE FIGURES

The drawings accompanying and forming part of this specification are included to depict certain aspects of the invention. A clearer impression of the invention, and of the components and operation of systems provided with the invention, will become more readily apparent by referring to the exemplary, and therefore nonlimiting, embodiments illustrated in the drawings, wherein identical reference numerals designate the same components. Note that the features illustrated in the drawings are not necessarily drawn to scale.

FIG. 1 is a diagram illustrating a dual-mode cooler in accordance with some embodiments.

FIGS. 2A, 2B and 2C are diagrams illustrating a dual-mode cooler system that includes a body cooler in accordance with some embodiments.

FIG. 3 is a diagram illustrating a medialess dual-mode cooler in accordance with some embodiments.

FIG. 4 is another example of a dual-mode cooler in accordance with some embodiments is shown.

FIGS. 5A-5B show the operation of a diverter.

FIGS. 6A-6B are diagrams illustrating body coolers in accordance with some embodiments.

FIG. 7 is a diagram illustrating the principle of operation of evaporative cooling systems.

FIG. 8 is a diagram illustrating an exemplary residential rooftop evaporative cooling system having a rigid, substantial cubic housing.

FIG. 9 is a diagram illustrating an exemplary portable evaporative cooling system having a rigid housing.

FIG. 10 is a diagram illustrating an exemplary evaporative cooling system having a collapsible/expandable housing in accordance with one embodiment.

FIG. 11 is a diagram illustrating an exemplary evaporative cooling system having a collapsible/expandable housing in the shape of a palm tree in accordance with one embodiment.

FIG. 12 is a diagram illustrating an exemplary evaporative cooling system having a collapsible/expandable housing that forms an inflatable tent or protective structure in accordance with one embodiment.

FIG. 13 is a diagram illustrating the connection of a first part of a housing to a second part of the housing, where the first part contains components of an evaporative cooling system and the second part forms an inflatable structure through which cooled air is distributed.

FIG. 14 is a diagram illustrating an exemplary portable evaporative cooling system in which the system's housing is connected to inflatable ducting that unrolls when the system is used and can be rolled up and stored with the evaporative cooling system when not in use.

FIG. 15 is a diagram illustrating an exemplary portable evaporative cooling system in which the system's housing is connected to removable/inflatable ducting that can be suspended from a roof or other structure to provide cooled air from above an area such as an outdoor dining patio.

FIG. 16 is a diagram illustrating an example of an evaporative cooling system that does not use evaporative media in accordance with some embodiments.

FIG. 17 is a diagram illustrating a control system in accordance with some embodiments.

FIG. 18 is a flow diagram illustrating a method in accordance with some embodiments.

FIGS. 19-20 are diagrams illustrating examples of an evaporative cooling system that operates without evaporative media in accordance with some embodiments.

### DETAILED DESCRIPTION

The invention and the various features and advantageous details thereof are explained more fully with reference to the non-limiting embodiments that are illustrated in the accompanying drawings and detailed in the following description. Descriptions of well-known starting materials, processing techniques, components and equipment are omitted so as not to unnecessarily obscure the invention in detail. It should be understood, however, that the detailed description and the specific examples, while indicating some embodiments of the invention, are given by way of illustration only and not by way of limitation. Various substitutions, modifications, additions and/or rearrangements within the spirit and/or scope of the underlying inventive concept will become apparent to those skilled in the art from this disclosure.

Disclosed herein are embodiments of a portable evaporative cooler that can be used to both prevent and treat heat injuries with unique modes of operation.

Generally, disclosed herein are embodiments of a portable evaporative cooler that can be used to both prevent and treat heat injuries with unique modes of operation. The disclosed coolers have two primary modes of operation:
First Mode (Convective Cooling): The first mode operates like a conventional evaporative cooler. Water is evaporated within an enclosure of the cooler to cool incoming air, which is typically dry and warm. The air exiting the cooler is cool and moist due to the evaporation of water inside the enclosure. This air can be directed into an area to cool the area and any objects or people in it via convection cooling.
Second Mode (Conductive Cooling/Artificial Sweating/Body Cooling): The second mode is adapted to cool an object, such as a body, by means of conduction. This mode is particularly useful for the treatment of heat injuries (hyperthermia), as it is necessary to cool the body as quickly as possible. Conductive cooling (evaporating water off the body) is known to be a more efficient and effective means to cool the object than convective cooling (directing already-cooled air over the body).

Mode 2 involves two primary actions: first, the system must be able to quickly turn off the evaporation process inside the cooler to allow warm air to exit as warm but unsaturated (dry) air; second, water is applied to the object (e.g., a human or animal body), and the exiting warm, dry air is then directed over the wetted object to evaporate the water off the object. The water evaporates directly off the object, drawing heat from the object via conduction. This process is loosely described as artificial sweating.

Water can be applied externally in various ways. For example, water can be applied by spraying through an external hose that is activated when the cooler ceases internal evaporation. Alternatively, a spray nozzle can be added to the exiting air stream that produces large droplets (in comparison to the small size droplets of mist) so that the water primarily hits and coats the object placed in the air stream. The intent is for the predominant water application and evaporation to occur on the object, not in the airstream. Using humidified or cooled air in Mode 2 is less effective because humidifying the air reduces its ability to evaporate water on the skin.

The body cooling concept can be applied to humans or animals, such as canines. For example, in a K9 cooling system, water would spray from underneath a dog bed, artificially sweating the dog.

To facilitate the quick transition necessary for emergency treatment, some embodiments employ a medialess evaporative cooler. Since there is no evaporative medium, the internal evaporation process can be stopped quickly, providing an advantage over traditional media-based coolers. The dual-mode system may include a control system, and the housing may be alternately expandable and contractible for easier storage and transport.

All conventional evaporative coolers have one mode of operation (purpose), which is providing cooled air. This cooled air is then used for either comfort or relief from heat (which may prevent heat injuries) through convection cooling. In other words, water is evaporated in the air to cool the air, and then the cooled air is directed to an area to cool the area and people or objects in the area.

The disclosed embodiments include another mode of operation which is adapted to cool an object (e.g., a body) by means of conduction. While embodiments are described in the context of objects such as a human body, the concepts described also apply to animals (e.g., canines), as one skilled in the art would understand. In this mode, the cooler does not evaporate water directly into the air, but instead directs uncooled air toward an object that has been wetted, so that the water, which is in contact with the object evaporates, thereby directly cooling the object. The evaporation of the water on the object also cools the previously uncooled air (typically to a lesser degree than in the conventional mode), so the now-cooled air may provide cooling to the same or other objects in the flow of the cooled air.

Conventional evaporative coolers cool air by evaporating water inside the evaporative cooler. Examples of conventional evaporative coolers are described in, for example, commonly-owned U.S. Pat. No. 10,113,758, entitled “Evaporative cooler,” and is fully incorporated by reference herein for all purposes. Warm unsaturated air enters the cooler which has water circulating therein (e.g., through evaporative media), becomes cool through evaporation of the water in the air, and then exits the cooler as cooled but water saturated air. This describes the first mode of operation of the disclosed coolers.

The second mode of operation of the disclosed coolers involves two parts. The first part is to be able to quickly turn off the evaporation process inside the cooler in order to allow warm air to enter the cooler and then exit as warm but also unsaturated air. The second part of this mode is that water is applied to the object (e.g., body) and then the warm unsaturated air is directed over the object to evaporate the water off the object. This cools the object through conductive means, which is a more efficient and effective means to cool the object than cooling the air and then directing the cooled air to/over the object.

The second mode of operation is particularly useful in the treatment of heat injuries. When a person or animal experiences hyperthermia (overheating of the body), it is necessary to cool the body as quickly as possible. As noted above, conductive cooling (evaporating water off the body) is more efficient and effective than convective cooling (directing already-cooled air over the body).

The most effective means for treating a heat injury is to reduce the core body temperature from its elevated temperature back to a normal temperature. In regular “operation”, human bodies regulate heat through a combination of convection, conduction (sweating) and radiation, with convection and conduction being the primary process. When the body gets hot, it sweats more to quickly cool down, but if the body gets too hot (i.e., experiences a heat injury), the body has essentially outpaced its own natural cooling ability and it is necessary to provide additional cooling through additional “artificial sweating.” This artificial sweating method helps alleviate the problems of vasoconstriction from ice baths/cold water emersion (see par. 4.2 of Appendix 2). Note that, while some animals, such as canines, regulate heat through other means (e.g., panting), the concepts described herein can be applied to canines and other animals to induce artificial sweating, even without sweat glands.

The conductive cooling process that occurs in the second mode of operation can loosely be described as artificial sweating. Rather than the body producing sweat on the skin, water is applied to the body by some means so that it can be evaporated by air flowing across the skin. The water can be applied to the body in a variety of different ways. For example, the water can be applied to the body by spraying the water through an external hose that is activated when the cooler ceases the evaporative cooling process. Alternatively, the water can be applied by adding a spray nozzle in the exiting air stream that produces large droplets of water (in comparison to the small size droplets in a mist) so that the water droplets primarily hit the object (body) placed in the air stream and coat the object. The coating of water on the object can quickly evaporate, as the temperature of the water is increased by contact with the warm body, and the water covers substantial surface area on the body.

While most conventional evaporative coolers have the ability to simply stop the flow/circulation of water across evaporative media, this does not necessarily stop the evaporation process, since some water remains on the evaporative media and the process of evaporation will continue until the water in the evaporative media is completely evaporated. Moreover, as long as the evaporation of the water in the evaporative media continues, the air flowing through the cooler will continue to be cooled and moisture will be added to the air. Both reduce the ability of the air exiting the cooler to evaporate water from a body outside the cooler.

The evaporation process within the evaporative cooler can be stopped more quickly if the cooler does not use evaporative media. Medialess evaporative coolers, for example, can use a mister or atomizer to spray very small droplets of water into an enclosure of the evaporative cooler so that the water droplets evaporate as they fall through the flowing air, rather than resting on an evaporative medium and evaporating from the medium. Examples of medialess evaporative coolers are described in, for example, commonly-owned U.S. Patent Application Publication No. 2023/0213222, entitled “EVAPORATIVE COOLER,” entitled “Evaporative cooler,” and is fully incorporated by reference herein for all purposes. Since there is no evaporative medium, the evaporation process can be stopped quickly. The ability to quickly stop the evaporation process inside the cooler can facilitate quickly treating a heat injury and thus provide an advantage over traditional coolers.

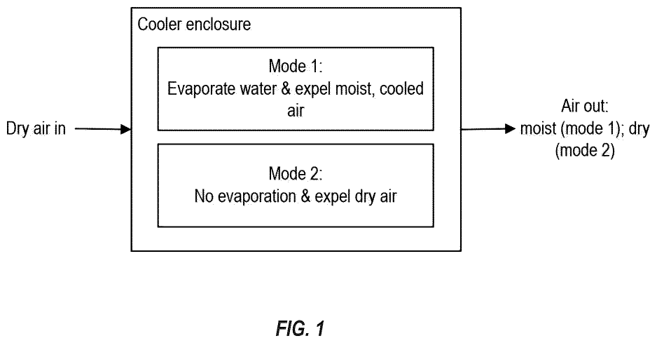

Referring to FIG. 1, a diagram is shown to illustrate a dual-mode cooler in accordance with some embodiments. The diagram depicts an evaporative cooler that can operate alternately in either of two modes. In the first mode, water is evaporated within an enclosure of the cooler to cool incoming air which is dry and warm. The air exiting the evaporative cooler in this mode is cool and moist due to the evaporation of water within the cooler enclosure as the air flows from an inlet to an outlet of the cooler. In the second mode, the cooler does not evaporate water into the air flowing through the enclosure, so the warm, dry air that is taken into the air inlet of the cooler is simply passed through to the air outlet of the cooler. The warm, dry air can then be directed to an object (e.g., a body) that has been wetted, so that the warm, dry air will evaporate the water on the object and cool the object via conduction.

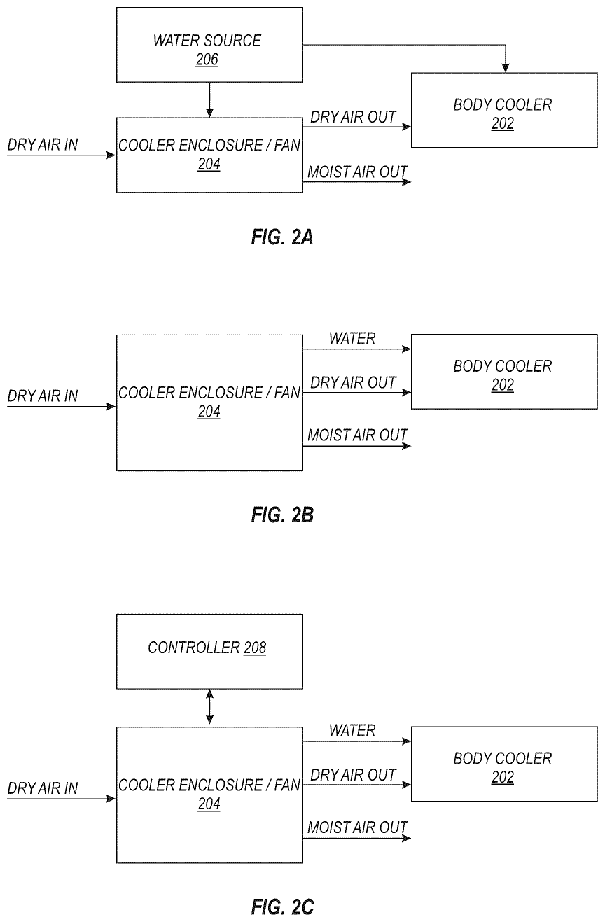

Referring to FIGS. 2A, 2B, and 2C, the diagrams are shown to illustrate a dual-mode cooler system that includes a body cooler 202 in accordance with some embodiments. As depicted in FIG. 2A, a cooler enclosure 204 has a fan that forces air through the enclosure 204. The fan draws warm, dry air into an air inlet and forces air out of the enclosure through one or more air outlets.

In the first mode, the air flowing through the enclosure 204 is cooled by evaporating water from a water source 206 into the air. This may be accomplished using evaporative media, or using a medialess water distribution system. The cooled air that exits the enclosure 204 can be directed into an area (e.g., a room, a tent, a shelter, etc.) to cool the area and any objects (e.g., people) in the area.

In the second mode, the evaporation of water into the air flowing through the enclosure is suspended or avoided so that the air exiting the enclosure 204 is not cooled, but is instead warm and dry. The warm, dry air is directed toward an object such as a body that is wetted so that the dry air will evaporate the water from the object, causing the object to be cooled.

In some embodiments, the dry air is directed to a body cooler 202 that is operatively connected to the cooler enclosure 204. The body cooler is also operatively connected to the water supply 206 so that the water can be sprayed on a body that is positioned within the body cooler 202. When the warm, dry air from the cooler enclosure 204 flows over the body, the water on the body evaporates, cooling the body.

FIG. 2B shows an embodiment that illustrates that the water source could be part of, or enclosed within the enclosure 204. In this example, the dual mode cooler system operates in the same manner as described above.

The cooler system may include a control system that receives inputs selecting either the first mode or the second mode and then directs water to either the cooler enclosure or the body cooler in accordance with the selected mode and provides either warm, dry air or cooled air at the outlet(s) of the enclosure in accordance with the selected mode. FIG. 2C shows an example of the cooler system shown in FIG. 2B, with the addition of controller 208. The operation of a cooler with a controller is described in more detail below.

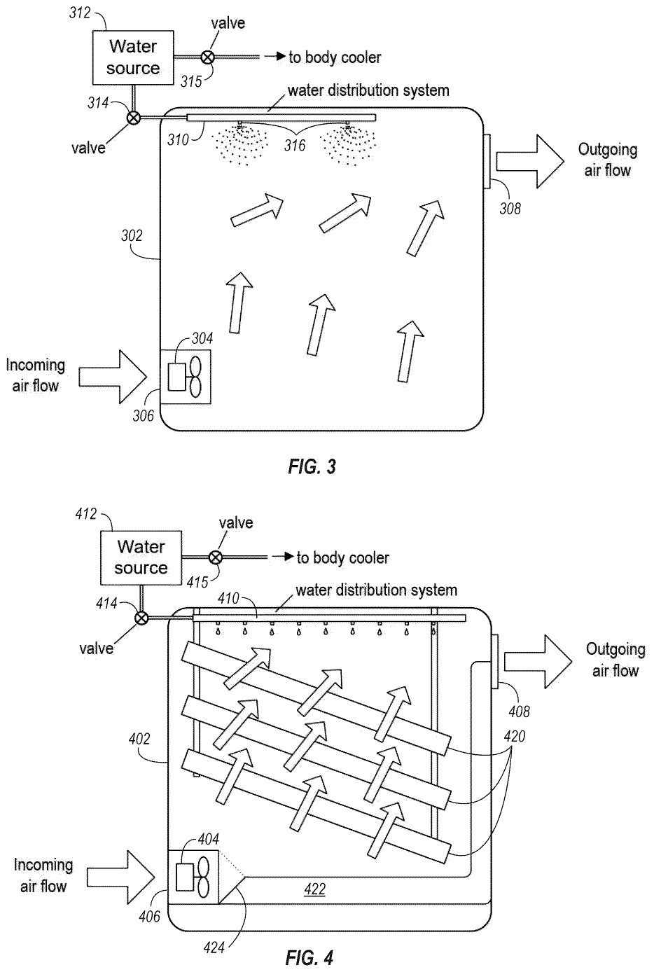

Referring to FIG. 3, a diagram is shown to illustrate a medialess dual-mode cooler in accordance with some embodiments. As depicted in this figure, the medialess dual-mode cooler comprises an enclosure 302 having an air inlet 306 and in air outlet 308. A fan 304 is positioned at air inlet 306 to draw air into the enclosure so that the air flows through the enclosure to outlet 308. The medialess dual-mode cooler also has a water distribution system 310 that is coupled to a water source 312 through a valve 314. A second valve 315 selectively allows water to flow to a body cooler which will be described in more detail below.

When the medialess dual-mode cooler is operated in a first mode, valve 314 is opened so that water from water source 312 is fed to water distribution system 310. Water distribution system 310 has one or more nozzles 316 that are configured to atomize or generate a fine mist of the water which is sprayed into the interior of enclosure 302. The water droplets are very fine and fall slowly through the enclosure, while air from inlet 306 flows generally upward toward outlet 308. As the water droplets fall through the air flow, the water is evaporated, cooling the air as it flows through the enclosure.

When the medialess dual-mode cooler is operated in a second mode, valve 314 is closed, so that no water flows to water distribution system 310. As a result, no droplets of water are injected into the interior of enclosure 302 and no evaporation occurs. As a result, the air flowing through the enclosure is not cooled and remains dry when it reaches outlet 308. This dry air is suitable to provide to a body cooling process (e.g., a wetted body (FIG. 6A), a body cooler (FIG. 6B)) to evaporate water that is in contact with the body, thereby cooling the body. As noted above, valve 315 may be opened to allow water to flow to the body cooler for the purposes of wetting the body.

As mentioned above with respect to FIG. 2C, the operation (including the control of the various modes, water volume, air flow, valves/ducting, etc.) of the cooler can be controlled by a controller. In some embodiments, the cooler system may receive inputs selecting mode 1 or mode 2 and directs water to either the cooler enclosure or the body cooler in accordance with the selected mode. The control system provides either warm, dry air or cooled air at the outlet(s). The control system can utilize multiple sensors, including temperature sensors, humidity sensors, airflow sensors, and pressure sensors, etc. (e.g., see FIGS. 17-20). These sensors provide measurement data to the controller, which processes the inputs and generates control signals to components such as the atomizer, inlet fan, or ducting. The controller adjusts operation to maximize cooling, adjust humidity, or minimize water waste. The controller may also receive user inputs for manual adjustments.

One of the advantages of the medialess dual-mode cooler is that the termination of water flow to the water distribution system immediately stops the injection of water droplets into the interior of enclosure 302 and therefore almost immediately stops the evaporation of water into the air flowing through the enclosure. (There may be some residual evaporation from previously injected water droplets.)

Referring to FIG. 4, a second example of a dual-mode cooler in accordance with some embodiments is shown. In this example, the system is not medialess, but instead uses evaporative media to cool the air flowing through the enclosure. Again, the system includes an enclosure 402 with an inlet 406 to allow air to flow into the enclosure and an outlet 408 to allow the air flow to escape the enclosure. Like the example of FIG. 3, a water source 412 is provided, with valves 414 and 415 to allow the water to flow either to a water distribution system 410 or a body cooling process.

In the example of FIG. 4, water from water distribution system 410 is distributed onto a set of evaporative media 420. Because the water is evaporated from the media, it is not necessary to generate the very fine mist that is generated in the medialess system of FIG. 3 (which falls slowly through the medialess enclosure). The water distribution system may instead simply drip water onto the media, which are suspended within the enclosure. As air is forced by fan 404 through the enclosure, the air passes through the evaporative media, and as water evaporates from the media, the air is cooled before flowing to outlet 408.

In the embodiment of FIG. 4, a bypass duct 422 is provided to allow air flowing from inlet 406 to be diverted around evaporative media 420 so that no evaporation is caused, and the dry air entering inlet 406 exits the duct as dry air at outlet 408. In some embodiments, outlet 408 may be split, with a first portion open to the interior of enclosure 402, and a second portion open to duct 422. A diverter 424 is positioned adjacent to fan 404 to direct the air alternately through enclosure 402 or duct 422.

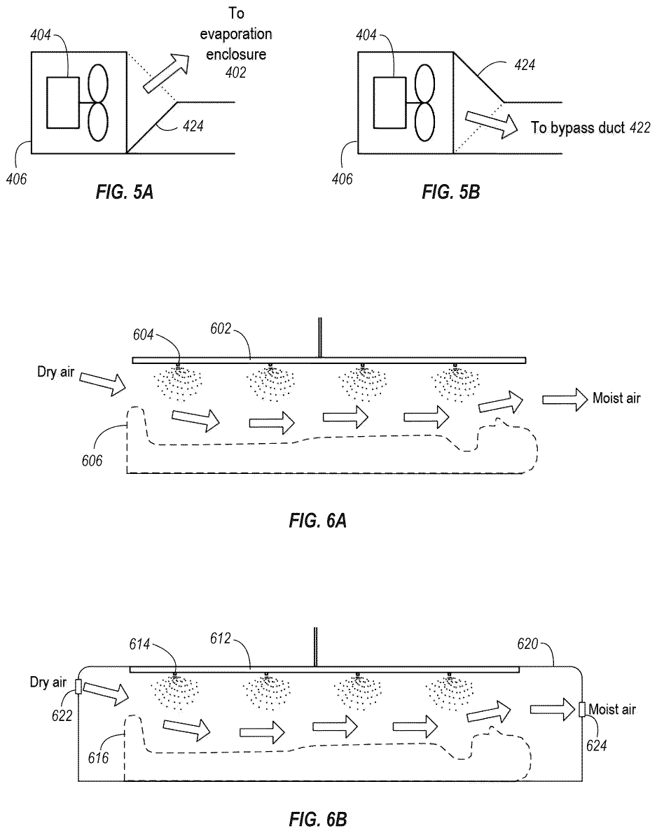

The operation of diverter 424 Is illustrated in FIGS. 5A and 5B. in FIG. 5A, diverter 424 is positioned to cause air from inlet 406 to be directed to enclosure 402. This corresponds to the first mode of operation in which the air is cooled as it flows through the evaporative media within enclosure 402. In FIG. 5B, diverter 424 is shown in a position that directs air from inlet 406 through bypass duct 422. This corresponds to the second mode of operation in which the dry air entering inlet 406 flows to outlet 408 without being evaporatively cooled.

Referring to FIGS. 6A and 6B, a pair of diagrams illustrating body coolers in accordance with some embodiments are shown. The embodiments depicted in these figures are coupled to a dry-air output of a dual-mode mode cooling system, e.g., as shown in FIGS. 3 and 4.

The example embodiment depicted in FIG. 6A includes a water distribution system 602 which is connected to a water source. The water source may be the same source that provides water to the water distribution system of the dual-mode cooling enclosure. As shown in FIG. 6A, water distribution system 602 includes multiple nozzles (e.g., 604) that are positioned above a body 606, in this example, to spray water on the body. In other examples, a body can be seated, standing, etc., as desired, while being cooled.

Dry air from the outlet of the dual mode cooler, operating in the second mode) is directed toward the body so that water sprayed by water distribution system 602 on the body will evaporate and thereby cool the body. In some embodiments, water distribution system 602 and the flow of dry air over the body are alternated, so that the water sprayed onto the body will settle on the body before the dry air is caused to flow over the body. In this manner, the evaporation of the water occurs while the water is in contact with the body, rather than evaporating in the air above the body.

Referring to FIG. 6B, an alternative body cooler is shown. In this embodiment, the body cooler again includes a water distribution system 612 with multiple nozzles, e.g., 614. The water distribution system sprays water onto the body so that dry air from the dual-mode cooler, operating in the second mode, can be directed over the body to evaporate the water that is in contact with the body.

The difference between the embodiment of FIG. 6B and the embodiment shown in FIG. 6A is that the embodiment of FIG. 6B includes an enclosure 620 that surrounds body 616. Dry air from the dual-mode cooler is directed to the interior of enclosure 620 through a dry air inlet 622. The air flows through the enclosure and, after evaporating water on the body, exits that enclosure via outlook 624. Enclosure 620 may, in alternative embodiments, have multiple air inlets and air outlets.

Enclosure 620 forces the air from the dual-mode cooler to flow within a constrained path along the body, as compared to the embodiment of FIG. 6A, in which the dry air is directed toward the body, but may flow away from the body. Enclosure 620 may thereby cause the air flowing over the body to evaporate more of the water on the body and more effectively cool the body.

As described above, the ability to quickly stop internal evaporation is beneficial. Conventional coolers using evaporative media cannot stop evaporation instantly because residual water remains on the media. The evaporation process will continue until the media dries out, which could take a significant amount of time. To overcome this, the evaporation process within the cooler can be stopped more quickly if the cooler does not use evaporative media. Medialess evaporative coolers utilize a mister or atomizer to spray very small water droplets into an enclosure. Since there is no evaporative medium, the evaporation process can be stopped almost immediately upon terminating water flow to the distribution system. The medialess dual-mode cooler is therefore much better suited to be dual mode than media-based systems.

Referring again to FIG. 3, the medialess dual-mode cooler operates in the two modes as follows:
In mode 1, valve 314 is opened, feeding water to system 310, which uses one or more nozzles 316 to atomize or generate a fine mist into the enclosure 302. As air flows upward, the water evaporates, cooling the air.
In mode 2, valve 314 is closed, immediately stopping water injection and preventing internal evaporation. The air flows through the enclosure uncooled and remains dry. A second valve, 315, may be opened to allow water to flow to a body cooling process (e.g., a body, a body in a body cooler) for the purpose of wetting the body.

Referring again to FIG. 4, the medialess dual-mode cooler operates in the two modes as follows:
Mode 1 (FIG. 5A): Diverter 424 directs air through enclosure 402 and media 420 for cooling.
Mode 2 (FIG. 5B): Diverter 424 directs air through bypass duct 422 to outlet 408 without being evaporatively cooled, thus exiting as dry air.

Valves 414 and 415 control water flow either to the media via system 410 (Mode 1) or to the body cooler (Mode 2).

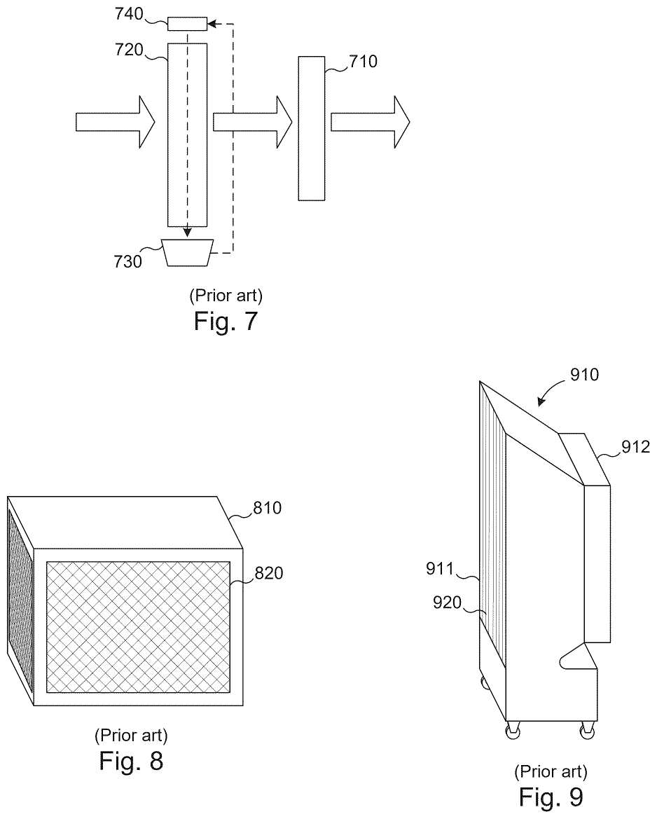

FIGS. 7-20 describe various cooling systems, configurations, controllers, applications, etc., to which the dual mode cooling concepts can apply.

One or more embodiments of the evaporative coolers, systems, applications, etc. are described below. It should be noted that these and any other embodiments described below are exemplary and are intended to be illustrative of the invention rather than limiting.

One exemplary embodiment of the present invention comprises an evaporative cooling system that has an expandable housing. When the system is not in use, the housing is collapsed to a smaller size so that it is more easily stored or transported. When the system is in use, the housing is expanded to a larger size, opening or expanding air flow pathways through the housing and the evaporative cooling media that are mounted on or in the housing. A water distribution system is provided to deliver water to the evaporative cooling media. A fan is also provided to draw air through the evaporative cooling media.

Referring to FIG. 7, a functional block diagram illustrating the principle of operation of evaporative cooling systems is shown. As depicted in this figure, a fan 710 draws air through an evaporative cooling medium 720. A water circulation system pumps water (as indicated by the dashed line) from a water source such as reservoir 730 to a manifold 740. Water flows out of manifold 740 onto evaporative cooling medium 720. The water moistens the medium and, as the air flows through the medium, the water evaporates and cools the air. Water that is not evaporated continues to flow downward through evaporative cooling medium 720, and is collected in reservoir 730. The collected water is then recirculated to manifold 740, from which it can again flow onto and through evaporative cooling medium 720.

Alternative embodiments may use varying components. For example, in the embodiment above, the fan is driven by an electric motor. The fan's motor may be powered by a battery, a generator or any other means. The fan may alternatively be driven by a combustion engine in other embodiments. The pump of the water distribution system is also driven by an electric motor in this embodiment. The pump motor may be powered by batteries, generators or other means. The water distribution system may alternatively be a non-recirculating system that distributes water from a source such as a garden hose or water storage unit, rather than pumping the water out of a reservoir which collects water that runs off from the evaporative cooling media. In a recirculating water distribution system, the reservoir may be a rigid structure, or it may be a flexible structure, such as a bladder.

Although not explicitly depicted in FIG. 7, the components of the evaporative cooling system are normally mounted to a rigid housing. The housing holds the evaporative cooling medium in position as the fan pulls air through it. The housing also supports the water distribution system so that it is properly positioned to direct the water onto the evaporative cooling medium.

Conventional evaporative cooling systems can be configured in various ways. One of the most common configurations is a residential rooftop installation. An exemplary system is depicted in FIG. 8. This illustration shows the housing 810 of the cooling unit with the evaporative cooling media 820 installed in the outer walls of the housing. Commonly, housing 810 has a simple, quasi-cubic shape and is constructed of stamped sheet metal. The housing typically has openings in either three or four sides, and the evaporative cooling media are installed in the openings. The bottom of the housing forms a reservoir for the water distribution system, and a pump is mounted within the housing to pump water from the reservoir to the tops of the evaporative cooling media. A fan is also mounted within the housing to draw air into the housing through the evaporative cooling media. The fan is coupled to a duct that extends through the rooftop into the building, so air that is drawn into the unit through the evaporative cooling media (thereby cooling it) and then blown into the home through the duct.

Another common configuration for a conventional evaporative cooling system is a portable unit. An exemplary portable evaporative cooling system is shown in FIG. 9. In this system, the housing 910 is a large structure that is commonly made of a rigid plastic such as PVC. The housing typically has a single large opening 911 on a first, inflow side of the unit, and a second, smaller opening 912 on the opposite, outflow side of the unit. Inflow opening 911 is typically larger because the evaporative cooling medium 920 is positioned within this opening, and it is desirable to maximize the area of the evaporative cooling medium in order to maximize the cooling effect of the medium. The unit's fan (which is not visible in the figure) is positioned within the second opening 912. The body of the housing typically tapers from the larger opening 911 to the smaller opening. When the fan operates, it pulls air through evaporative cooling medium 920 in opening 911 and blows the air out of opening 912. As with the residential unit, a pump is provided to draw water out of a reservoir which is integrated into the lower portion of the housing and deliver the water to the top of the evaporative cooling medium. The water flows downward through the evaporative cooling medium, and the air flowing through the evaporative cooling medium is cooled by evaporation of the water. Wheels are attached to the housing below the reservoir to allow the unit to be rolled from one location to another.

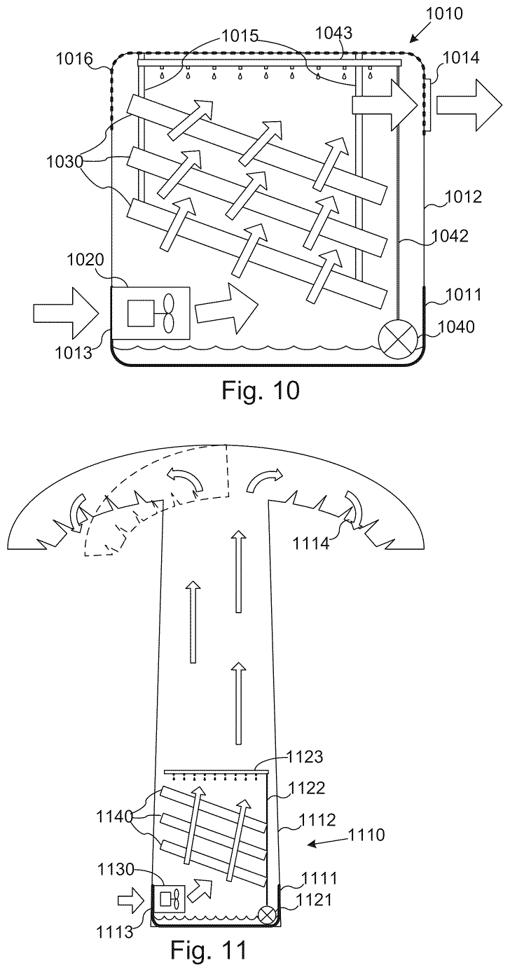

Referring to FIG. 10, a diagram illustrating the structure of an exemplary embodiment of the present invention is shown. In this embodiment, an evaporative cooling system 1000 includes a housing or enclosure 1010, a fan 1020, evaporative cooling media 1030, and a water distribution system. The primary difference between this embodiment and conventional evaporative cooling systems is that housing 1010 is expandable and collapsible, rather than being a rigid structure that does not change its shape. During operation, cooling system 1000 is in an expanded position that allows air to flow through pathways within the housing from the inlet to the outlet. These air flow pathways occupy a certain amount of empty volume. In a conventional evaporative cooling system, the rigid, unchanging shape of the housing maintains this empty volume, whether the system is in use or not. In the present systems, the housing can be collapsed when not in use, thereby reducing or eliminating the empty volume within the housing. By reducing or eliminating the empty space in the housing, the overall volume occupied by the system is reduced, which makes the system easier to store and/or transport.

In the embodiment of FIG. 10, housing 1010 includes a rigid or semi-rigid lower portion 1011 and a flexible upper portion 1012. Lower portion 1011 may be formed from PVC or other plastics. Lower portion 1011 forms a tray or shallow tub that functions as a reservoir for water that flows through the evaporative cooling media. The reservoir formed in the lower portion may have a layer of thermal insulation 1017. This tray may also serve as all or part of the shipping and storage container for the system when not in use. In an alternative embodiment, the tray portion of the system may also be flexible (e.g., made of a waterproof fabric) that can be folded or collapsed. Upper portion 1012 may be constructed from a fabric or similar flexible material. The lower edge of upper portion 1012 is connected to the upper edge of lower portion 1011. Fan 1020 is mounted to the lower portion 1011 of the housing at an inlet to the housing. Fan 1020 forces air into the housing, generating a positive pressure differential between the interior and exterior of the housing. This positive pressure differential (where the air pressure inside the housing is greater than the air pressure outside the housing) causes the housing to move to its expanded position when the fan is operating. Effectively, the fan inflates the housing. By contrast, conventional evaporative cooling systems typically position the fan at the air outlet so that operation of the fan creates a negative pressure differential that pulls air into the housing. With a negative pressure differential, the housing must be rigid in order to keep it from collapsing. It should be noted that alternative embodiments of the present invention may use a collapsible rigid housing. For instance, the housing may have rigid walls that fold into a collapsed position for storage and transport, or into an expanded position for operation of the cooling system. When a rigid collapsible housing is used, a positive internal pressure differential may not be necessary, and the fan may be positioned at an outlet in the housing.

The embodiment of FIG. 10 includes multiple evaporative cooling media 1030. These media may, for example, be fibrous pads or other suitable media. The media are arranged within the housing so that when the housing is in its expanded position, there is space between the media. The media are positioned between the fan at the inlet 1013 to the housing and the outlet 1014 of the housing so that when the fan is operating, the air is forced through each of the media before exiting the housing. Because the air interacts with multiple media, the maximum cooling effect is achieved (compared to a single media interaction like conventional coolers).

The water circulation system includes a pump 1040 that draws water from the reservoir in the lower portion 1011 of the housing and pumps this water through one or more tubes 1042 to a manifold 1043 that delivers the water to evaporative cooling media 1030. The water may be sprayed, dripped, or otherwise delivered to the evaporative cooling media. The portion of the water that flows through the media and is not evaporated is collected in the reservoir and is then recirculated. In some alternative embodiments, passive mechanisms may be used to deliver the water to the evaporative cooling media. For example, a wicking mechanism (which uses capillary action to draw the water from the reservoir) may be used, or an inlet fan may create water droplets that are blown onto the evaporative cooling media. In one embodiment, the housing is configured so that, after passing through the evaporative cooling media, the speed of the cooled air is reduced to a level at which water droplets are allowed to fall out of the air before it exits the housing. This prevents the system from producing an undesirable mist, and it also enables the system to provide effective cooling for a longer period of time since more water is retained within the housing.

The media may be hinged or otherwise configured so that when the housing is in the collapsed position, the media are more closely positioned (e.g., stacked on top of each other) and require less space in the housing. In the embodiment of FIG. 10, each of evaporative cooling media 1030 is supported by a plurality of straps or cables 1015 that extend downward from the top of the housing. When the housing is moved to the collapsed position, the top of the housing moves downward, lowering the straps so that the evaporative media rest on each other within lower portion 1011 of housing 1010. The components of the water distribution are also configured to move with the expansion and contraction of the housing. In one embodiment, the pump is mounted in a fixed position in the lower portion of the housing and flexible tubing coupled to the pump extends to the top of the housing. A manifold (e.g., perforated tubing that is attached to the top of the housing) receives water from the pump through the flexible tubing and distributes the water across the evaporative cooling media.

It is not unusual for mold growth and bacterial growth to occur in moist environments such as evaporative cooling systems. The present system may therefore incorporate means to prevent mold growth. In one embodiment, an ozone generator 1050 is positioned within the housing at the air inlet and/or in the water collection section of the system. When ozone is generated in the air, it flows through the housing and through the evaporative cooling media with the air as it is being cooled. In some embodiments, the water that is circulated through the distribution system and evaporative cooling media is ozonated. In both cases, the ozone in the air and/or the ozonated water is circulated through the system, thereby disinfecting the housing and evaporative cooling media. Other means to combat mold and bacteria may also be used.

The housing may have many different configurations. As described in connection with FIG. 10, the Housing has a rigid lower portion and a flexible fabric upper portion. In one alternative embodiment, the upper portion may comprise several rigid but movable components. For instance, the vertical walls of the housing may be hinged so that they fold together (accordion-style) when the housing is in its compact state. The walls components may alternatively be movable with respect to each other (e.g., telescoping) so that they occupy different positions in the housing's operating and compacted states. In another embodiment, the housing may have rigid upper (416) and lower (411) portions with a flexible intermediate portion connected between them. In this embodiment, when the housing is in its expanded state, the intermediate portion is extended between the upper and lower portions, and when the housing is in its compacted state, the intermediate portion collapses to allow the upper and lower portions to come together to form a suitcase-like shell. Alternatively, the system could employ a collapsible frame that supports the top or body portions of the housing. Any or all of these embodiments may include features such as wheels and stowable handles on the housing and to facilitate movement or transport of the systems. The housing components may include rigid, semirigid and flexible components in any combination.

While the housing depicted in FIG. 10 is generally rectangular, alternative embodiments may have many different shapes. In particular, the housing can include a flexible fabric portion that takes on shapes which would not be practical in a conventional system. For example, as depicted in FIG. 11, one embodiment of an evaporative cooling system uses a housing that has the form of a palm tree. In this embodiment, the housing 1110 includes a lower portion 1111 and an upper portion 1112. Lower portion 1111 is similar to the lower housing portions described above, in that it is made of a substantially rigid material that forms a reservoir for a water circulation system, and has a pump 1121 for the water circulation system mounted to it. A fan 1130 is also mounted to lower housing portion 1111 at an inlet 1113 to force air into housing 1110.

The upper portion 1112 of the housing in this embodiment is made of a lightweight fabric (e.g., nylon). Upper housing 1112 has the shape of a tree (e.g., a palm tree), including a trunk portion that is attached at its lower end to lower housing portion 1111, and one or more branch/leaf portions that extend outward from the trunk portion. Several evaporative cooling media 1140 are positioned within the trunk portion of the housing. A water distribution tube 1122 extends from pump 1121 to a water distribution manifold 1123 that is positioned at the top of the evaporative cooling media.

When fan 1130 is operated, it forces air from inlet 1113 into housing 1110. The air pressure inside the housing causes the flexible housing to inflate and take on the tree shape. The air that is forced into the housing flows upward through evaporative cooling media 1140 and is cooled by evaporation of the water in/on the media. The air continues to flow upward through the trunk portion of the housing and into the branch/leaf portion(s). Air outlets (e.g., 1114) are provided in the branch/leaf portions so that the cooled air is distributed to the area around the housing, particularly under the branch/leaf portion of the housing.

When the fan is not being operated, it no longer produces a positive pressure differential between the interior and exterior of the housing, so the upper fabric portion of the housing deflates. It should be noted that the evaporative cooling media 1140 and the water distribution system (particularly the tubing and manifold portions) are movably mounted within housing 1110 so that they move into a compact position when the upper portion of the housing deflates. This allows the overall volume of the evaporative cooling system to be reduced, making it easier to store and/or transport the system.

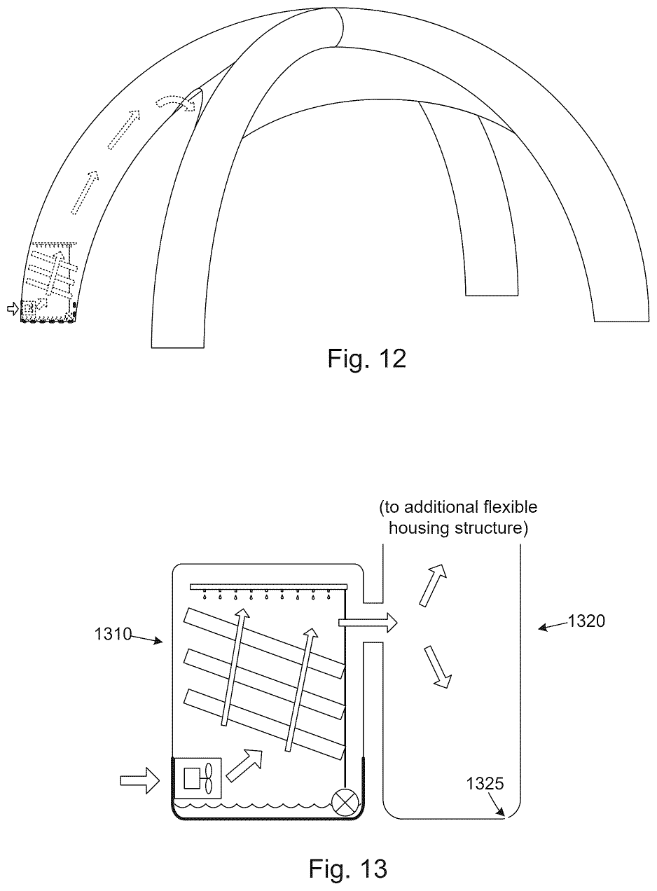

Another alternative embodiment is shown in FIG. 12. In this embodiment, an evaporative cooling system includes a housing that forms a tent or other type of shelter when the system is in operation. The housing includes an upper portion made of a flexible (e.g., nylon) fabric that is inflated when the fan of the evaporative cooling system is turned on. Air that is cooled by the system flows out through air outlets (in an upper part of the tent portion so that the cooled air is directed onto the area under the tent. When the fan is turned off, the portion of the housing forming the tent collapses, enabling the convenient storage and/or transportation of the system.

Components of the system such as the fan, air inlet, reservoir, water distribution system, and the like may, for example, be located in the bottom of one of the legs of the tent, similar to the location at the bottom of the trunk portion of the system of FIG. 11. Alternatively, these components may be located in a portion of the housing that forms a separate structure that is adjacent to the flexible portion of the housing and forces cooled air into the flexible portion of the housing. This configuration is illustrated in FIG. 13, which shows the fan, air inlet, reservoir, water distribution system, etc. in a first portion 1310 of the housing. The cooled air from portion 1310 is forced by the air pressure inside this portion of the housing into the flexible tree/tent portion 1320 of the housing. Portion 1320 of the housing may include a weep hole 1325 to allow mist which collects in this portion of the housing to escape from the housing.

In another alternative embodiment, multiple fans may be used, where at least one of the fans'primary function is to inflate the structure, while at least one of the other fans is used to provide the cooling air to the structure. In yet another alternative embodiment, a conventional inflatable tent or similar structure can be converted to a cooling system by inserting an evaporative cooling element (e.g., evaporative cooling media and water distribution subsystem) between the fan and the inflatable tent structure of the conventional system.

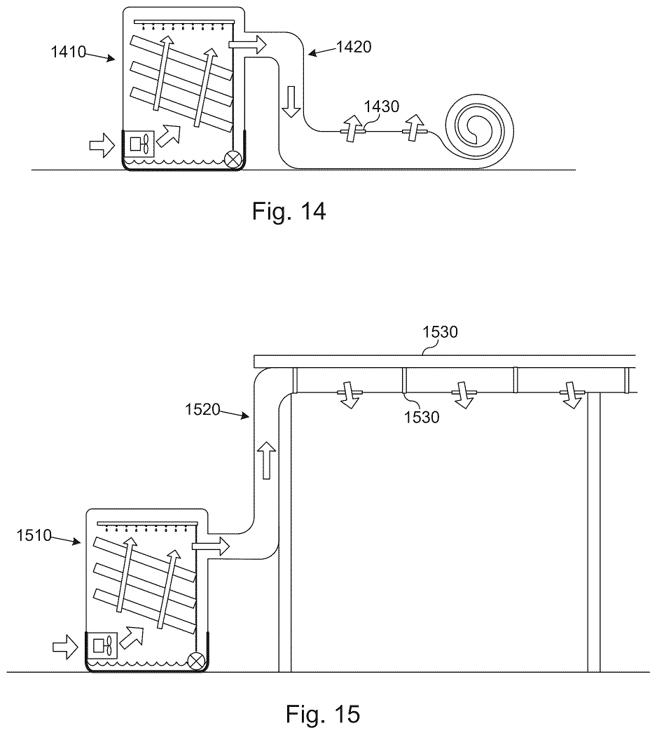

Referring to FIG. 14, a diagram illustrating an alternative portable evaporative cooling system is shown. In this embodiment, the system's housing 1410 is connected to inflatable ducting 1420 that unrolls when the system is used and can be rolled up and stored with the evaporative cooling system when not in use. The ducting can be made of a lightweight fabric that takes up very little space when it is rolled up, potentially allowing the ducting to be stored with the evaporative cooling system. The ducting has several outlets 1430 along its length that allow the cooled air to be distributed through the duct to a desired area.

Referring to FIG. 15, a diagram illustrating another alternative embodiment is shown. In this embodiment, a portable evaporative cooling system 1510 is connected to removable/inflatable ducting 1520 that can be suspended from a roof 1530 or other structure. This system provides cooled air from above the desired area without impeding access to the area being cooled, such as an outdoor dining patio. The ducting can be made of a fabric that can easily be supported by straps 1540 or other means connected to the roof. The ducting can easily be installed as desired to meet the cooling requirements for the area. When the ducting is not in use, it is deflated and can easily be removed.

While the embodiments described above distribute water onto evaporative media to provide cooling of the air that is circulated through the systems, some alternative embodiments do not require evaporative media for this purpose. These alternative embodiments use an atomizer to generate a very fine water mist. Because the water droplets of the mist are very fine, they do not fall quickly to the bottom of the enclosure, but are instead effectively suspended within the enclosure by the air that flows through the enclosure. The increased surface area of the very small droplets also allows the water to evaporate more quickly. Still further, since the evaporative media are not required, the system may be capable of collapsing into a smaller volume than embodiments that use evaporative media.

A mist generator, mister, or mist source, etc., can take many forms. For the purposes of this description, the term atomizer will be used to refer to various devices and techniques for generating a mist. For example, an atomizer can be a water distribution system that sprays water into a moving airstream so the moving air breaks up the stream into discrete droplets. In another example, an atomizer can be a water distribution system that sprays water under pressure to break up the stream into discrete droplets at the nozzle as it enters the airstream. In another example, an atomizer can be vibrating devices that uses vibrations, like ultrasonic misters, that eject discrete droplets of water away from the water surface. In another example, an atomizer can be spinning devices, like controlled droplet applicators (CDAs) that use centrifugal forces to separate a water stream into discrete droplets. Various other devices or techniques can also be used to generate a mist, as one skilled in the art would understand.

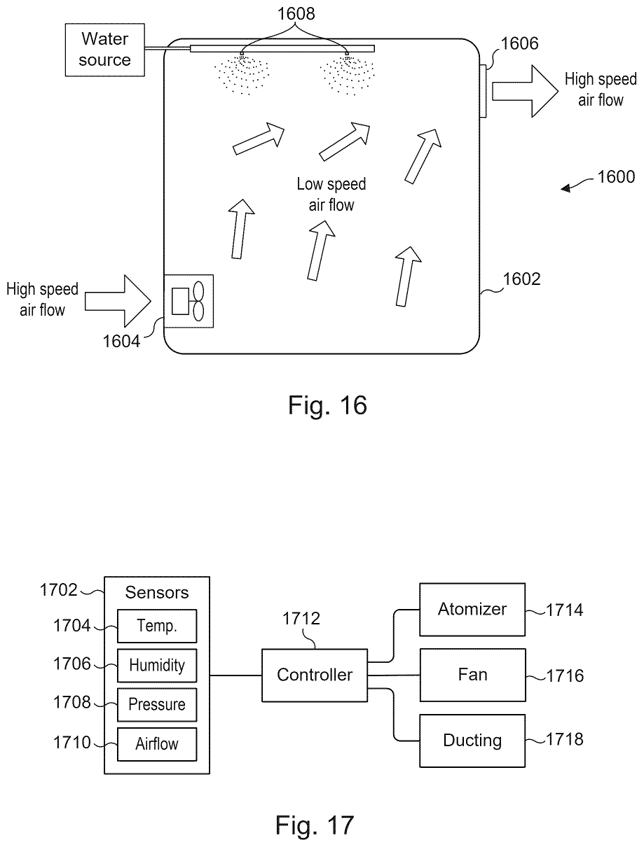

Referring to FIG. 16, an example of an evaporative cooling system that does not use evaporative media is shown. In this embodiment, the enclosure 1602 of cooling system 1600 includes an inlet fan 1604 that forces air into the enclosure and a cooled air outlet 1606 from which the air exits the enclosure. As in the embodiments described above, the pressure of the air forced into enclosure 1602 may be used to expand the enclosure from its collapsed position. In alternative embodiments, however, the enclosure may be expanded manually without the assistance of the forced air.

At the top of enclosure 1602 are a set of atomizers or misters 1608. Atomizers 1608 may be ultrasonic atomizers, ultrafine spray misters, or any other suitable mechanism to generate the desired size of water droplets in the mist. Atomizers 1608 receive water from a water source and generate a fine mist of water droplets which are sprayed into the enclosure. While atomizers 1608 may be located at various different positions within enclosure 1602, it is generally beneficial to position the atomizers near the top of the enclosure since gravity will tend to draw the droplets downward.

It is further generally beneficial to position inlet fan 1604, or more specifically the air inlet to the enclosure, near the bottom of enclosure 1602 with the air outlet near the top of the enclosure. This generates an upward component of the air flow through the enclosure, which tends to drive the water droplets upward, against the pull of gravity. The air flow's effect of suspending the water droplets (or slowing the fall of the droplets through the enclosure) is dependent upon a number of different factors, such as the size of the water droplets, the rate of air flow through the enclosure and the speed of the air as it flows through the enclosure.

If the droplets are smaller, the droplets may completely evaporate before reaching the floor of the enclosure. In this case, it may not be necessary to provide any means for collecting and/or recirculating the water. By eliminating the need for water collection and/or recirculation means, the cost, complexity and size of the system may be reduced. It should be noted, however, that if the atomized droplets are small enough to completely evaporate, but the volume of water that is atomized is less than the maximum amount that could be evaporated under the prevailing conditions, the system will not achieve the maximum cooling that is possible under the conditions. In other words, more water could have been evaporated, and greater cooling could have been achieved. Some embodiments therefore implement controls to sense relevant conditions and adjust the operation of the cooling system to try to maximize the cooling provided by the system.

Referring to FIG. 17, a functional block diagram illustrating a control system in accordance with some embodiments is shown. In this embodiment, multiple sensors are employed to measure a variety of different parameters relevant to the operation of the cooling system. Sensors 1702 include temperature sensors 1704, humidity sensors 1706, airflow sensors 1708, pressure sensors 1710, etc. The sensors may measure parameters both within the enclosure and external to the enclosure. The types and placement of sensors included in the figure are intended to be illustrative, rather than limiting, and other types of sensors may be used as well. Conversely, not all of these sensors are required, and some embodiments may use fewer sensors than are depicted in the figure. Other types of sensors that could be used include, for example, water level sensors (for water supplies or reservoirs), vent/door sensors (for sensing whether a vent, door, valve, etc. is open or closed), mist/water droplet sensors, etc., as one skilled in the art would understand.

Sensors 1702 are coupled to provide measurement data to a controller 1712. Controller 1712 processes each of these inputs from the sensors and generates control signals that are output to add atomizer 1714, inlet fan 1716 and ducting 1718. The control signals are adapted to adjust the operation of these components to adjust the operation and/or performance of the system (e.g., to maximize the cooling of the system, adjust the humidity of the output air, etc.). For example, if a signal received from a humidity sensor indicates that the humidity of the air external to the cooling system is high, a lower percentage of the water sprayed by the atomizer may evaporate as the droplets fall through the cooling system enclosure. The controller may therefore reduce the amount of water that is provided to the atomizer for generation of the mist within the enclosure. On the other hand, if the humidity sensor indicates that the humidity of the external air is very low, a higher percentage of the water sprayed into the enclosure by the atomizer may evaporate, so the controller may increase the amount of water provided to the atomizer.

Another benefit of a controllable system is that it can minimize the amount of water wasted as unevaporated droplets or mist in the airflow once the air is saturated. This also benefits in that people or objects downstream in the airflow do not get wet from the unevaporated excess water. Another benefit of the controllable system is that it allows for an evaporative cooler to be used as humidifier in a deliberate and controllable manner, which is not currently possible with evaporative coolers.

Controller 1712 similarly uses inputs from the other sensors to generate control signals which are output to atomizer 1714, inlet fan 1716, and any other components that may be controlled by controller 1712 (e.g., valves, recirculating pumps, controllable ducts, vents, doors, etc.). It should be noted that controller 172 may use any suitable the algorithms or methodologies to generate the control outputs. These algorithms are not described in detail herein because the particular algorithm that is used in a given embodiment is not important to the patentability of the embodiment. Note that the controller can be physically located in different places, as desired. For example, a controller can be a part of the cooling system, as well as being located remotely, while still being operatively connected to the sensors and various other components of the cooling system. A controller can be connected to the components of a cooling system via a wired connects as well as wirelessly.

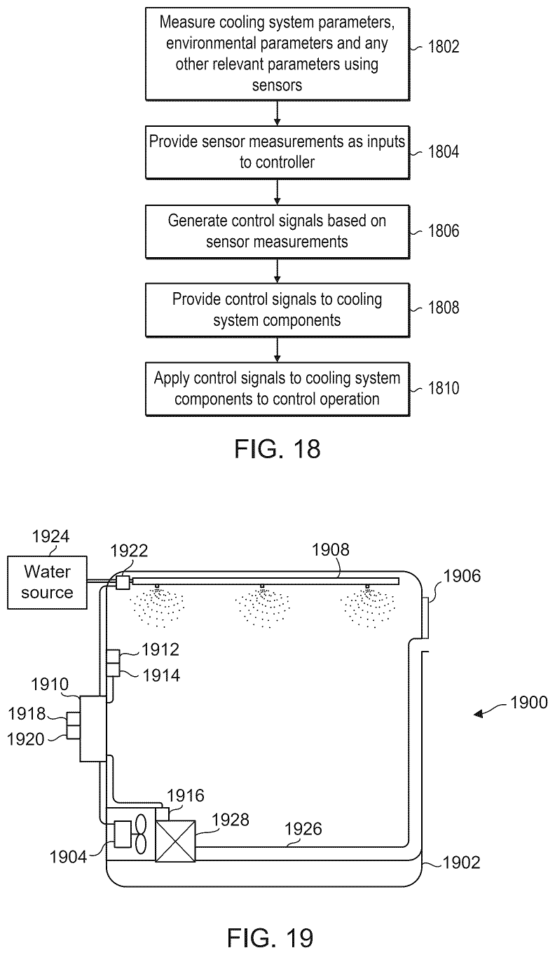

Referring to FIG. 18, a flow diagram illustrating a method in accordance with one embodiment is shown. In this method, measurements of one or more parameters or conditions relevant to the operation of the cooler are taken by one or more corresponding sensors (1802). These parameters may include, for example, cooling system parameters (e.g., air flow, fan speed, water flow), environmental parameters (e.g., humidity and temperature) and any other relevant parameters.

These measurements of the various parameters are provided by the sensors as inputs to the controller (1804). In addition to the sensor signals that are provided to the controller, one or more user inputs may be provided to the controller to enable the user to manually adjust the operation of the cooling system. For example, the system may have manual controls to adjust a level of cooling (low/medium/high), a fan speed, a target temperature, a target humidity level, or some other operating parameter.

The controller then uses the received inputs representative of the operating parameters to generate a set of control signals (1806). The controller then provides the generated control signals to one or more of the components of the cooling system (1808), such as the inlet fan or the atomizer. The control signals are applied to the respective ones of the cooling system components to control the operation of these components (1810), such as changing the speed of the inlet fan to adjust the flow rate of air through the cooling enclosure, changing the flow rate of water to the atomizer to adjust the volume of water mist that is generated by the atomizer, opening/closing/adjusting doors or vents or bypasses to affect air flow, changing the operation of pumps/valves (e.g., for different water sources) based on sensed conditions such as water levels, etc. The operation of the cooling system can also be changed based on mist droplets (size density, etc.). For example, if there is excess mist (e.g., exiting the outlet), the mister can be controlled to reduce the amount of mist produced. Or, if the droplets are too large, the mister can be adjusted to form smaller droplets.

Referring to FIG. 19, another example of an evaporative cooling system that operates without evaporative media is shown. In this embodiment, cooling system 1900 has an enclosure 1902 with an inlet fan 1904 at or near the bottom of the enclosure that forces air into the enclosure and an air outlet 1906 at or near the top of the enclosure that allows the air to exit the enclosure. An atomizer 1908 at or near the top of the enclosure is fed by a water source and is configured to generate a water mist within the enclosure. As air flows from the inlet to the outlet, it flows upward against the falling mist so that the mist evaporates and cools the air before it exits the enclosure.

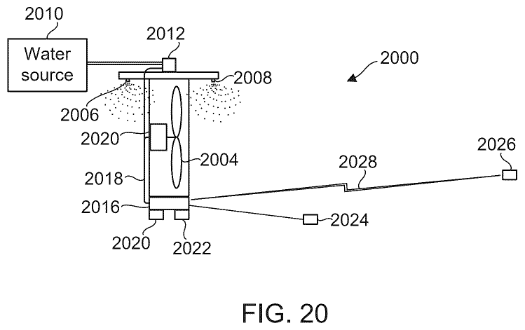

While the embodiment of FIG. 19 uses an enclosure within which the water is evaporated into the air flow, alternative embodiments need not use such an enclosure. For example, one alternate embodiment may use an open fan with an atomizer or mister that injects water droplets into the air flow created by the fan. The operation of the fan (e.g., fan speed) and atomizer/mister (e.g., water flow/output) are controlled based on the outputs of sensors (e.g., humidity, temperature) that are provided to a control system that generates control signals to adjust the operation of the fan and atomizer/mister. An example of such an embodiment is provided in FIG. 20.

In this embodiment, a controller 1910 is coupled to atomizer 1908 and inlet fan 1904 to control operation of these components. In this case, controller 1910 controls a valve 1922 that controls the flow of water from water source 1924 to atomizer 1908. Controller 1910 also controls a ducting valve 1928 that can be adjusted to direct airflow through ducting 1926, as well as through the interior of enclosure 1902. Ducting valve 1928 determines the percentage of the airflow that flows through ducting 1926 and the percentage airflow that flows through enclosure 1902 and can thereby affect the amount of cooling or humidification that is achieved by evaporating water into the air flowing through the enclosure.

Controller 1910 is further coupled to a set of sensors that are located internal to and external to enclosure 1902. In this embodiment, the sensors include internal temperature sensor 1912 and humidity sensor 1914, airflow sensor 1916, and external temperature sensor 1918 and humidity sensor 1920. Although not explicitly depicted in the figure, controller 1910 may also have user controls (e.g., temperature settings, cooling level settings, fan settings, etc.) that allow a user to select various options for the operation of the cooling system. Controller 1910 uses the inputs from the sensors and user controls to compute control outputs for atomizer 1908 and fan 1904 (or any other controllable components of the cooling system). The user controls can take any form desired, such as a control panel, an electronic interface or touch screen, a web interface, etc.

Referring to FIG. 20, an alternative embodiment of an evaporative cooling system that operates without evaporative media is shown. In this embodiment, cooling system 2000 operates without an enclosure. The cooling system has a fan 2004 that is mounted on a structure that may leave the area around the fan almost completely open, or it may have a shroud that surrounds the periphery of the fan to help direct the airflow through the fan.

A set of atomizers or misters 2006, 2008 (which are at the top of the cooling system in this figure, but may be positioned in alternative locations in other embodiments) receive water from a water source 2010 through a controllable valve 2012. Controllable valve 2012 and fan motor 2014 are controlled by control signals received via control lines 2018 from a controller 2016.

Controller 2016 is also connected to a set of sensors that provide feedback to the controller as to the cooling effects of the system. In this embodiment, two sensors 2020, 2022 are connected to the cooling system at or near fan 2004 to measure local parameters, such as the temperature and humidity in the immediate vicinity of the cooling system. A third sensor 2024 may be positioned remotely from the cooling system to measure one or more conditions at a distance from the cooling system. Sensor 2024 is connected to controller 2016 by a physical communication line. This embodiment also includes a remote sensor 2026 that is coupled to controller 2016 by a wireless communication channel, which may allow the sensor to be conveniently positioned to sense environmental conditions without being hindered by a physical connection to the controller. This type of sensor may be particularly useful in scenarios where it is desired to achieve a desired temperature or humidity level in a room or area, rather than simply maximizing the cooling effect of the evaporative cooler.

There may be various alternative placements of the sensors depending on what output or control of the system is required to produce intended results. For example, in the example embodiments, the sensors may be placed in the inlet airstream, outlet airstream, in the immediate vicinity of the cooling system, and/or in the area that is being cooled or humidified. Note that sensors can be placed anywhere desired, including internally or externally to a cooler. In addition, note that sensors can be placed in any type of cooler, for example, coolers with enclosures and with media, coolers with enclosures but no media, coolers without enclosures, etc. If sensors are placed in the outlet airstream, characteristics of the outlet airstream can be controlled. If sensors are placed in the local area, then the control of the local area can be controlled. If sensors are placed in an area remote from the cooling system, then the control of the larger area around the system can be controlled.

There may be various alternative atomizer-based embodiments. For example, one alternative embodiment comprises an evaporative cooling system having an enclosure with an air inlet that enables air to flow into the enclosure and an air outlet that enables air to exit the enclosure. An atomizer is positioned within the enclosure, where the atomizer receives water from a water source and generates a water mist within the enclosure. Air from the air inlet is circulated through the enclosure and is cooled by evaporation of the mist before being provided at the air outlet.

In some embodiments, the atomizer and the air outlet are positioned at or near the top of the enclosure and the air inlet is positioned at or near the bottom of the enclosure, so that the mist falls through the enclosure, while flows upward through the enclosure.

In some embodiments, the evaporative cooling system includes a controller coupled to the atomizer and/or the inlet fan, wherein the controller generates one or more control signals that control operation of the atomizer and/or inlet fan. The controller may be configured to control a rate at which the atomizer injects the mist into the enclosure and/or to control a rate at which the fan causes air to flow through the enclosure.

In some embodiments, the evaporative cooling system includes one or more sensors coupled to the controller, where each of the sensors senses a corresponding parameter associated with operation of the evaporative cooling system and provides a corresponding sensor signal as an input to the controller, and where the controller generates the one or more control signals based at least in part on the received sensor signals. The one or more sensors may be configured to sense humidity, temperature, pressure, airflow, or other parameters associated with operation of the system. The sensors may be configured to sense parameters corresponding to conditions both internal and external to the enclosure. User controls may also be coupled to the controller, where each user control provides a corresponding user control signal as an input to the controller, and where the controller generates the control signals based at least in part on the received user control signals.

In some embodiments, the inlet fan generates high-speed air flow through the inlet to the enclosure and high-speed air flow at the outlet from the disclosure. The larger volume of the enclosure allows the air to flow more slowly upward from the lower portion of the enclosure to the upper portion of the enclosure. Since the air flows more slowly through the volume of the enclosure, the mist generated by the atomizers has more time to evaporate. The low-speed air flow also allows unevaporated droplets to fall out of the air, which enables the system to produce air at the outlet, which is cooled, but which does not contain water droplets that can cause people using the system to feel sticky or uncomfortable.

In some embodiments, the enclosure has a portion that is alternately expandable and contractible, where when the first portion of the enclosure is contracted, the enclosure occupies a first volume, and when the first portion of the enclosure is expanded, the enclosure occupies a second volume that is greater than the first volume. The fan may be configured to force air into the enclosure to create a positive pressure differential between the interior of the enclosure and the exterior of the enclosure, thereby expanding the enclosure.

An alternative embodiment comprises a method for providing evaporative cooling, where an evaporative cooler enclosure is provided, air is drawn into the enclosure through an air inlet, a water mist is generated by an atomizer within the enclosure, and the air is circulated through the enclosure, where it is cooled by evaporation of the mist, so that cooled air is provided from an air outlet of the enclosure.

In some embodiments, the method includes sensing one or more parameters associated with operation of the evaporative cooling system, providing corresponding sensor signals as inputs to a controller, generating one or more control signals based at least in part on the received sensor signals, and providing the control signals to at least one of the atomizer and a fan that circulates the air through the enclosure. The sensed parameters may comprise one or more of: humidity; temperature; pressure; and airflow. The method may also include providing one or more user control signals to the controller and generating control signals based at least in part on the received user control signals, and providing the generated control signals to at least one of the atomizer and the fan.

It should be noted that the foregoing atomizer-based embodiments may include features that are disclosed in the previously described evaporative-media-based embodiments. For example, atomizer-based embodiments may use expandable/contractable enclosures, and may be coupled to inflatable structures such as those shown in FIGS. 11-15.

While the present invention has been described with reference to particular embodiments, it should be understood that the embodiments are illustrative and that the scope of the invention is not limited to these embodiments. Many variations, modifications, additions and improvements to the embodiments described above are possible. It is contemplated that these variations, modifications, additions and improvements fall within the scope of the invention as detailed within this disclosure.

Embodiments may include, for example: an evaporative cooling system comprising: an enclosure; an air inlet that enables air to flow from an exterior of the enclosure to an interior of the enclosure; an air outlet that enables air to flow from the interior of the enclosure to the exterior of the enclosure; an atomizer positioned within the enclosure, wherein the atomizer receives water from a water source and generates a mist of the water within the enclosure; and wherein air from the air inlet is circulated through the enclosure and thereby cooled by evaporation of the mist, and the cooled air is provided at the air outlet.

Such an evaporative cooling system, wherein the atomizer is positioned at a top of the enclosure so that the mist falls through the enclosure; wherein the air inlet is positioned at a bottom of the enclosure; wherein the air outlet is positioned at the top of the enclosure; wherein the air from the air inlet flows upward through the enclosure to the air outlet.

Such an evaporative cooling system, further comprising a controller coupled to the atomizer, wherein the controller generates one or more control signals that control operation of the atomizer.

Such an evaporative cooling system, wherein the controller is configured to control a rate at which the atomizer injects the mist into the enclosure.

Such an evaporative cooling system, further comprising a fan positioned at the air inlet, wherein the controller generates one or more control signals that control operation of the fan.

Such an evaporative cooling system, wherein the controller is configured to control a rate at which the fan causes air to flow through the enclosure.

Such an evaporative cooling system, further comprising one or more sensors coupled to the controller, wherein each of the sensors senses a corresponding parameter associated with operation of the evaporative cooling system and provides a corresponding sensor signal as an input to the controller, wherein the controller generates the one or more control signals based at least in part on the received sensor signals.

Such an evaporative cooling system, wherein at least one of the one or more sensors is configured to sense humidity.

Such an evaporative cooling system, wherein at least one of the one or more sensors is configured to sense temperature.

Such an evaporative cooling system, wherein at least one of the one or more sensors is configured to sense pressure.

Such an evaporative cooling system, wherein at least one of the one or more sensors is configured to sense airflow.

Such an evaporative cooling system, wherein at least one of the one or more sensors is configured to sense parameters of droplets in the airstream, including, for example, droplet/mist size, velocity, direction, quantity, etc. This enables the ability to dial back (or increase) water injection, based on sensed conditions.

Such an evaporative cooling system, wherein the one or more sensors comprise a plurality of sensors, wherein a first portion of the plurality of sensors is configured to sense parameters corresponding to conditions internal to the enclosure and a second portion of the plurality of sensors is configured to sense parameters corresponding to conditions external to the enclosure.

Such an evaporative cooling system, further comprising one or more user controls coupled to the controller, wherein each of the user controls provides a corresponding user control signal as an input to the controller, wherein the controller generates the one or more control signals based at least in part on the received user control signals.

Such an evaporative cooling system, wherein the enclosure has a first portion that is alternately expandable and contractible, wherein when the first portion of the enclosure is contracted, the enclosure occupies a first volume, and when the first portion of the enclosure is expanded, the enclosure occupies a second volume that is greater than the first volume.

Such an evaporative cooling system, wherein the fan is configured to force air into the enclosure and thereby create a positive pressure differential between the interior of the enclosure and the exterior of the enclosure, thereby expanding the enclosure.

Another embodiment may include a method for providing evaporative cooling in an evaporative cooling system, the method comprising: providing an evaporative cooler enclosure; drawing air into the enclosure through an air inlet; providing water to an atomizer positioned within the enclosure, wherein the atomizer receives water from a water source and generates a mist of the water within the enclosure; circulating the air through the enclosure, wherein the air is cooled by evaporation of the mist; and providing the cooled air from the enclosure through an air outlet.

Such a method, further comprising sensing one or more parameters associated with operation of the evaporative cooling system; providing corresponding sensor signals as inputs to a controller; generating, by the controller, one or more control signals based at least in part on the received sensor signals; and providing the control signals to at least one of the atomizer and a fan that circulates the air through the enclosure.

Such a method, wherein sensing the one or more parameters associated with operation of the evaporative cooling system comprises sensing one or more of: humidity; temperature; pressure; and airflow and water droplet size and quantity.

Such a method, further comprising providing one or more user control signals to a controller; generating, by the controller, one or more control signals based at least in part on the received user control signals; and providing the control signals to at least one of the atomizer and a fan that circulates the air through the enclosure.

As noted above, the housings in embodiments of the present invention can alternately be in a compacted state or an expanded state. For the purposes of this disclosure, terms such as “expanded”, “higher volume”, “increased-volume”, and the like may be used interchangeably to describe the expanded state in which the system operates. The compacted state that the system may be in when it is not operating may be referred to using interchangeable terms including “compacted”, compact”, “reduced-volume”, “lower volume”, and the like.

Another embodiment may include a method for providing evaporative cooling in an evaporative cooling system, the method comprising: drawing air through a fan; providing water to an atomizer positioned before or after the fan, wherein the atomizer receives water from a water source and generates a mist of the water within the airstream; and providing the cooled air from fan.

Although the invention has been described with respect to specific embodiments thereof, these embodiments are merely illustrative, and not restrictive of the invention as a whole. Rather, the description is intended to describe illustrative embodiments, features and functions in order to provide a person of ordinary skill in the art context to understand the invention without limiting the invention to any particularly described embodiment, feature or function, including any such embodiment feature or function described in the Abstract or Summary. While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention.

Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

It will also be appreciated that one or more of the elements depicted in the drawings/figures can be implemented in a more separated or integrated manner, or even removed or rendered as inoperable in certain cases, as is useful in accordance with a particular application. Additionally, any signal arrows in the drawings/figures should be considered only as exemplary, and not limiting, unless otherwise specifically noted.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, product, article, or apparatus.

Furthermore, the term “or” as used herein is generally intended to mean “and/or” unless otherwise indicated. For example, a condition A or B is satisfied by any one of the following: A is true (or present) and B is false (or not present), A is false (or not present) and B is true (or present), and both A and B are true (or present). As used herein, a term preceded by “a” or “an” (and “the” when antecedent basis is “a” or “an”) includes both singular and plural of such term, unless clearly indicated within the claim otherwise (i.e., that the reference “a” or “an” clearly indicates only the singular or only the plural). Also, as used in the description herein and throughout the meaning of “in” includes “in” and “on” unless the context clearly dictates otherwise.

Additionally, any examples or illustrations given herein are not to be regarded in any way as restrictions on, limits to, or express definitions of, any term or terms with which they are utilized. Instead, these examples or illustrations are to be regarded as being described with respect to one particular embodiment and as illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” “in one embodiment.”

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

Generally then, although the invention has been described with respect to specific embodiments thereof, these embodiments are merely illustrative, and not restrictive of the invention. Rather, the description is intended to describe illustrative embodiments, features and functions in order to provide a person of ordinary skill in the art context to understand the invention without limiting the invention to any particularly described embodiment, feature or function, including any such embodiment feature or function described. While specific embodiments of, and examples for, the invention are described herein for illustrative purposes only, various equivalent modifications are possible within the spirit and scope of the invention, as those skilled in the relevant art will recognize and appreciate.

As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

What is claimed is:

1. A multi-mode cooling apparatus for selectively cooling an area via evaporative air cooling or cooling a body via external water evaporation, the apparatus comprising:
an enclosure;
an air inlet that enables air to flow from an exterior of the enclosure to an interior of the enclosure;
an air outlet that enables air to flow from the interior of the enclosure to the exterior of the enclosure;
an internal water distribution system, wherein the water distribution system is configured in a first mode of operation to receive water from a water source and generate a source of water in an interior of the enclosure, and is configured in a second mode of operation to be disabled or diverted, thereby allowing a body wetted by water to be cooled by the air outlet;
a water source configured in the first mode of operation to supply water to the water distribution system, and is configured in the second mode of operation to suspend the supply of water to the internal water distribution system; and
a fan configured to move ambient air through the enclosure, from the air inlet toward the air outlet, forming an air stream, such that in the first mode of operation the air stream is cooled and humidified inside the enclosure before exiting the air outlet, and in the second mode of operation unsaturated dry air is expelled from the air outlet and directed over a wetted body.

2. The apparatus of claim 1, wherein the enclosure of the multi-mode cooling apparatus is medialess.

3. The apparatus of claim 2, wherein an absence of evaporative media allows the evaporative air cooling process to be substantially suspended immediately upon disabling the water distribution system.

4. The apparatus of claim 1, further comprising a bypass duct configured to fluidly connect the air inlet to the air outlet, wherein the bypass duct allows the air stream to be diverted around the open interior of the enclosure to prevent evaporation in the second mode of operation.

5. The apparatus of claim 4, further comprising a diverter positioned adjacent to the fan, wherein the diverter is configured to cause the air stream to flow through the open interior of the enclosure during the first mode of operation, and cause the air stream to flow through the bypass duct during the second mode of operation.

6. The apparatus of claim 5, further comprising evaporative media positioned within the interior of the enclosure, wherein the evaporative media is configured in the first mode of operation to receive water from the water source and humidify the air stream flowing through the enclosure.

7. The apparatus of claim 1, further comprising a body cooler enclosure external to the cooling apparatus, wherein the enclosure is configured to surround the body and receive the unsaturated dry air expelled from the air outlet to maximize evaporation of water on the body.

8. The apparatus of claim 1, further comprising a controller operatively coupled to the multi-mode cooling apparatus, wherein the controller is configured to receive an input selecting either the first mode or the second mode of operation, and based on the selected mode, directs water to either the water distribution system or the external body cooling process.

9. The apparatus of claim 1, wherein the enclosure has a portion that is alternately expandable and contractible, such that when the portion is contracted, the enclosure occupies a first volume, and when the portion is expanded, the enclosure occupies a second volume that is greater than the first volume.

10. The apparatus of claim 1, further comprising evaporative media positioned within the enclosure, wherein the evaporative media is configured in the first mode of operation to receive water from the water source and humidify the air stream flowing through the enclosure.

11. A medialess multi-mode cooling apparatus for selectively cooling an area via evaporative air cooling or cooling a body via external water evaporation, the apparatus comprising:
an enclosure;
an air inlet that enables air to flow from an exterior of the enclosure to an interior of the enclosure;
an air outlet that enables air to flow from the interior of the enclosure to the exterior of the enclosure;
an atomizer positioned within the enclosure, wherein the atomizer is configured in a first mode of operation to receive water from a water source and generate a mist of the water distributed throughout a substantial volume of the interior of the enclosure, and is configured in a second mode of operation to be disabled;
a water source configured in the first mode of operation to supply water to the atomizer, and is configured in the second mode of operation to supply water to a body cooling process external to the enclosure; and
a fan configured to move ambient air through the substantial volume of the interior of the enclosure, from the air inlet toward the air outlet, forming an air stream, such that in the first mode of operation the air stream is cooled and humidified inside the enclosure before exiting the air outlet, and in the second mode of operation unsaturated dry air is expelled from the air outlet and directed over a body wetted by water supplied by the water source.

12. The apparatus of claim 11, wherein an absence of evaporative media allows the evaporative air cooling process to be substantially suspended immediately upon disabling the atomizer.

13. The apparatus of claim 11, further comprising a body cooler enclosure external to the cooling apparatus, wherein the enclosure is configured to surround the body and receive the unsaturated dry air expelled from the air outlet to maximize evaporation of water on the body.

14. The apparatus of claim 11, further comprising a controller operatively coupled to the multi-mode cooling apparatus, wherein the controller is configured to receive an input selecting either the first mode or the second mode of operation, and based on the selected mode, directs water to either the atomizer or the external body cooling process.

15. The apparatus of claim 11, wherein the enclosure has a portion that is alternately expandable and contractible, such that when the portion is contracted, the enclosure occupies a first volume, and when the portion is expanded, the enclosure occupies a second volume that is greater than the first volume.

16. A method of treating heat injury or cooling an area using a dual-mode cooler, the method comprising:
operating a fan to drive an air stream through an enclosure from an inlet to an outlet;
selecting a first mode of operation wherein water is distributed into the air stream within the enclosure to evaporatively cool the air stream before it exits the outlet; and
selecting a second mode of operation wherein the distribution of water into the air stream within the enclosure is suspended or bypassed while the fan continues to operate, thereby expelling an unsaturated air stream from the outlet configured to conductively cool a wetted body external to the enclosure.

17. The method of claim 16, wherein the enclosure of the dual-mode cooler is medialess.

18. The method of claim 17, wherein an absence of evaporative media allows the evaporative air cooling process to be substantially suspended immediately upon disabling the distribution of water.

19. The method of claim 16, further comprising providing a bypass duct configured to fluidly connect the air inlet to the air outlet, wherein the bypass duct allows the air stream to be diverted around the open interior of the enclosure to prevent evaporation in the second mode of operation.

20. The method of claim 19, further comprising causing the air stream to flow through the bypass duct during the second mode of operation.

21. The method of claim 16, further comprising providing evaporative media positioned within the enclosure, wherein the evaporative media is configured in the first mode of operation to receive the distributed water and humidify the air stream flowing through the enclosure.
